(19) United States
(12) Patent Application Publication
(10) Pub. No.: US 2009/0024159 A1
(43) Pub. Date: Jan. 22, 2009

(54) Tourniquet with wooden windlace

(76) Inventors: Joseph Patrick Nee, Calhan, CO (US); Joseph Robert Farrell, Philadelphia, PA (US)

Correspondence Address: JOSEPH P. NEE, 23590 MCDANIELS RD, CALHAN, CO, 80808, US

(21) Appl. No.: 12/220,262

(22) Filed: Jul. 21, 2008

Related U.S. Application Data
(60) Provisional application No. 60/951,199, filed on Jul. 21, 2007.

(51) Int. Cl. A61B 17/132

(52) U.S. Cl. 606/203

(57) ABSTRACT

A tourniquet system design for providing self-use in emergencies and ma occurrences. The user can simple and quickly slip the tourniquet into place, fix it to adjust tightly against the affected limb. The user can then twist the handle to apply pressure and stop potentially life threatening loss of blood.

### CROSS-REFERENCE TO RELATED APPLICATIONS

No. 60/951,199.

### FEDERALLY SPONSORED RESEARCH

None.

### SEQUENCE LISTING

None.

### BACKGROUND OF INVENTION

The present invention relates to a tourniquet apparatus for controlling the loss of blood from an injured limb. Specifically the invention relates to a tourniquet apparatus, which can be rapidly self-operated.

Many tourniquets for constricting the flow of blood in injured limbs currently in the art employ clamps or buckles for securing the tourniquet, which may loosen if bumped or fail to provide sufficient pressure to occlude the flow of blood from the affected limb. Others utilize designs, which make them slow, complicated or expensive to manufacture, thus what is needed in the art is a tourniquet, which is simple, self-operated and capable of use under extreme conditions such as combat situations.

### SUMMARY

The present invention relates to a tourniquet system, which may be operated by a single user under extreme life threatening emergencies, which employs sufficient and adjustable pressure to restrict the loss of blood from an injured limb.

In one aspect of the invention the tourniquet system includes a slotted handle and a strap. The slot permits a loop formed by the strap to pass through and encircle the affected limb. The handle of the tourniquet apparatus twists to sufficiently tighten.

In one aspect of the inventions, the tourniquet system includes a chord passed through a hole in one end of the handle to secure it once tightened.

In another arrangement, the strap has a slide able shield, which permits the handle a twisting motion without tissue damage. Various other modifications will be apparent to and readily made by those skilled in the art without departing from the scope and spirit of the invention.

### DRAWING DESCRIPTIONS 1-2-3-4-5-6

The accompanying drawings, which are incorporated into and form part of the specifications, illustrate several embodiments of the present invention and together with the description serve to explain the principles of invention.

### DRAWINGS

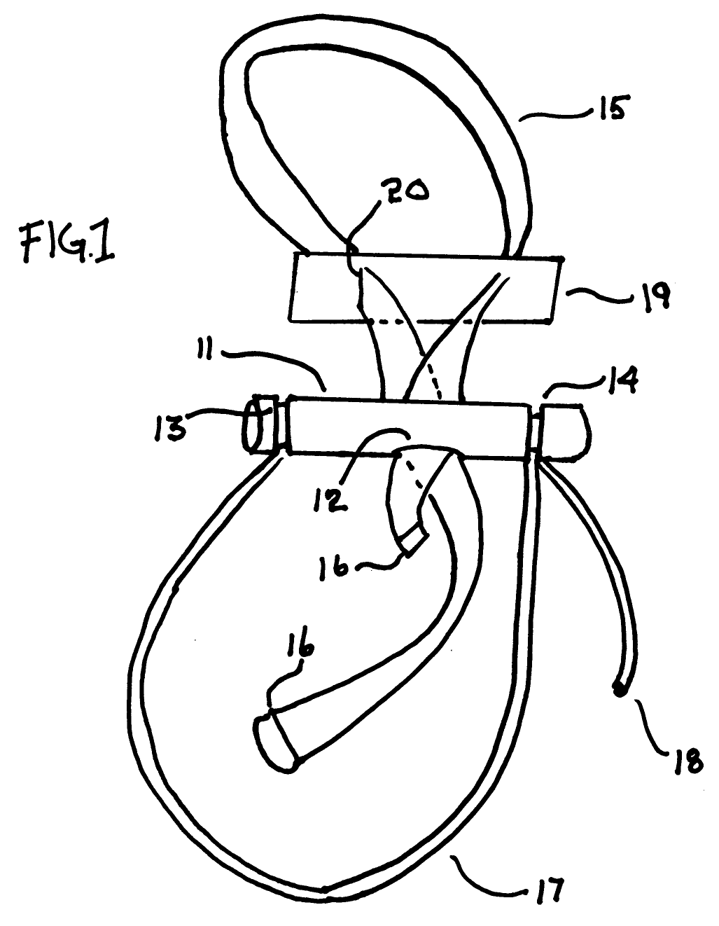

FIG. 1 is a perspective view of an embodiment of tourniquet according to the present invention.

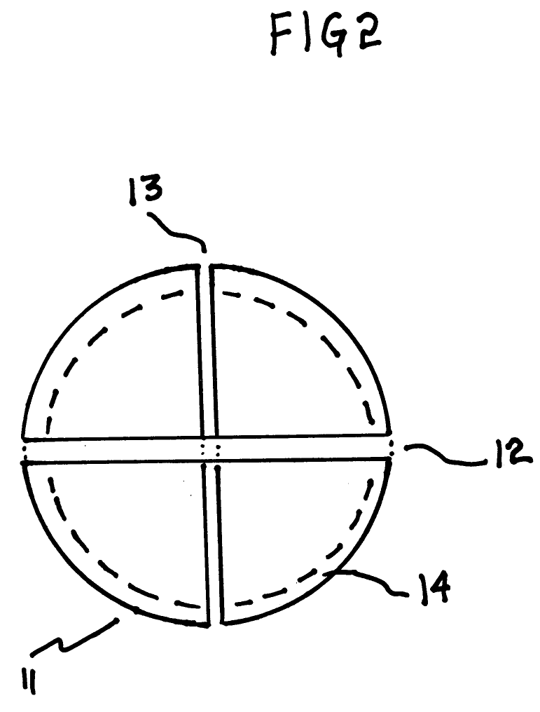

FIG. 2 is a cross section of handle (11) showing the angled relationship between cord hole (13) and strap slot (12).

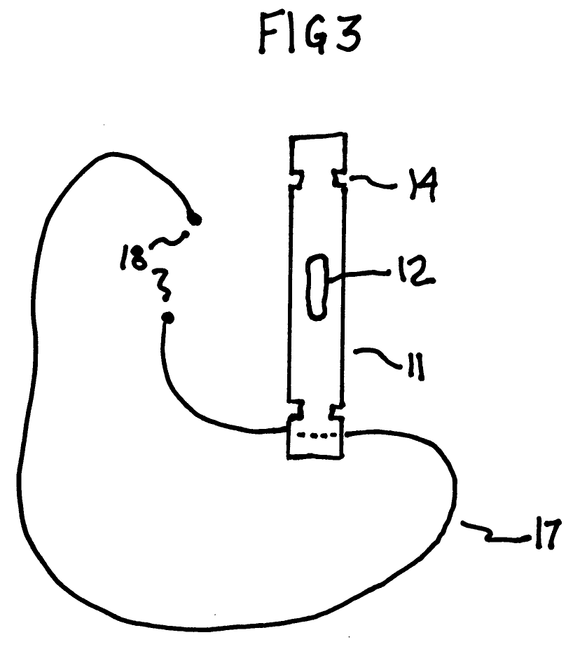

FIG. 3 is a perspective view of handle (11) showing strap slot (12), cord hole (13) and cord (17) from another view.

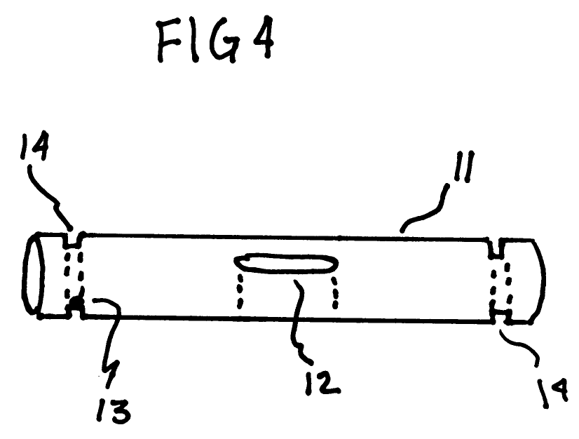

FIG. 4 is a perspective view of handle (11) showing cord hole (13), cord slot (14) and strap slot (12).

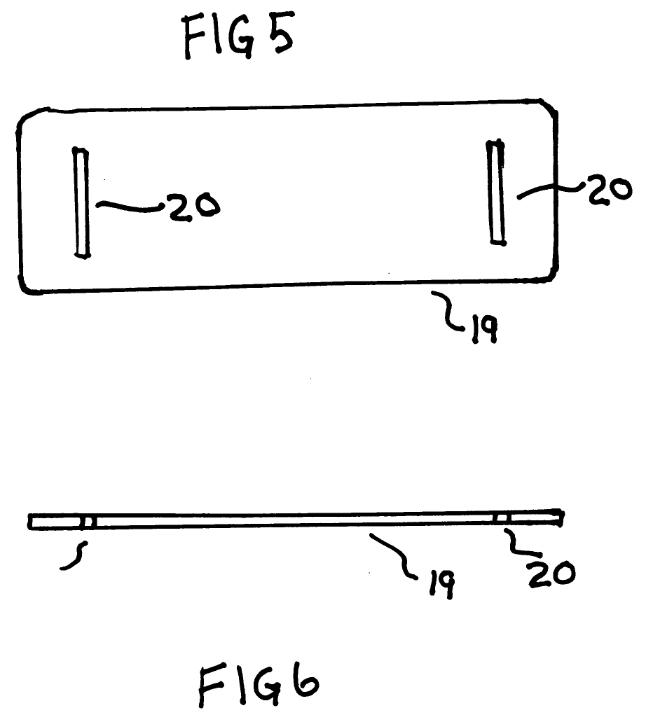

FIGS. 5 & 6 are a perspective view of skin shield (19) showing strap slots (20).

### DETAILED DESCRIPTION

FIG. 1 is a perspective view of the embodiment of the present invention showing a loop end of the nylon-webbing strap (15) threadable pulled through the handle (11) by means of strap slot (12). It further shows strap snubs (16) ends a means to prevent said strap (15) from pulling through strap slots (12). FIG. 1 also shows a cord (17) threaded through the cord hole (13) and cord snubs (18) a means to retain cord (17) threadably passed through said handle (11). FIG. 1 depicts skin shield (19) threadably affixed to the apparatus by means of shield slots (20) through which strap (15) passes in opposing directions.

FIG. 2 is a perspective view of the apparatus handle (11) a cross sectional view of strap slot (12) and cord hole (13) through which strap (15) and cord (17) are threadably pulled.

FIG. 3 a side perspective view of handle (11) depicting the approximate relation of strap slot (12) and cord hole (13) to each other and handle (11).

FIG. 4 is a perspective view of handle (11) showing cord hole (13), cord slot (14) and strap slot (12).

FIG. 5 is a perspective view from above said skin shield (19) showing the approximate relationship of shield slots (20) on the skin shield (19). Said skin shield (19) is comprised of flexible plastic or rubber like substance capable of forming to an arm or leg.

FIG. 6 is a perspective view from the front of said invention a skin shield (19), handle (11), and strap (15) threadably pulled through shield slots (20) to form appendage loop and proper placement close to handle (11).

### REFERENCE NUMERALS

11 handle
12 strap slot
13 cord hole
14 cord slot
15 strap
16 strap snubs
17 cord
18 cord snubs
19 skin shield
20 shield slots

### FIG. 1-2-3-4-5-6

1 entire view
2 handle cross section
3 handle side view
4 handle side view
5 skin shield top view
6 skin shield side view

### Operation

In operation one uses the embodiment of the invention by first pulling the looped end of strap (13) such that the strap snubs (16) on either end of the strap (15) are pulled against the handle (11) at the strap slots (12):
(1) The injured appendage is thereby placed through the loop created by strap (15).
(2) One strap snub (16) end is pulled in such a manner as to cause skin shield (19) to fix snug against appendage (21) and handle (11).
(3) Handle (11) is thence opened vertiginously either clockwise or counterclockwise until sufficiently occluding arterially or venial exsanguinations.
(4) Cord (17) is thence pulled in such a manner and direction around the limb as to maintain pressure on the handle (11) and prevent said handle (11) from untwisting Cord (17) is thence looped around handle (11) on either end in the cord slot (14) to lock it into place.

1. A tourniquet system comprising: a strap; a cord; a handle with a slot and a hole and cord slots at both ends wherein said strap is threadable through the slot and said cord is threadable through the hole and secures in cord slots at either end of the handle.

2. Tourniquet system of claim 1 wherein said strap is comprised of nylon webbing.

3. Tourniquet system of claim 1 wherein said strap comprises a length of between 36 inches and approximately 40 inches.

4. Tourniquet system of claim 1 wherein said cord is comprises of nylon cord.

5. Tourniquet system of claim 1 wherein said cord comprises a length of approximately 36 inches and ⅛ inch in diameter.

6. Tourniquet system of claim 1 said handle is comprised of wood.

7. Tourniquet system of claim 1 wherein said handle is comprises a diameter of 1 inch and a length of 5.75 inches.

8. Tourniquet system of claim 1 said handle comprises a slot 3/16 inches in width and 1 inch in length located longitudinally at a point mid way from each end.

9. Tourniquet system of claim 1 wherein said strap has at each of its ends snubs by means of triple folding back ends and being secured.

10. Tourniquet system of claim 1 cord has at each end snubs by means of knots.

11. Tourniquet system of claim 1 wherein said skin shield is comprised of thin flexible plastic or rubber like material with a width sufficient to prevent strap from contacing skin, said shield has two slots for threadably accepting the strap.
